(19) United States
(12) Patent Application Publication
(10) Pub. No.: US 2010/0261684 A1
(43) Pub. Date: Oct. 14, 2010

(54) Pharmaceutical combination of medication to be used by patients presenting with an acute coronary syndrome so as to stop the process and prevent a myocardial infarction

(76) Inventor: Jacques Benoit, Montreal (CA)

Correspondence Address: JACQUES BENOIT, 7013 JEAN MILOT, MONTREAL, QC, H1M3G4, CA

(21) Appl. No.: 12/386,008

(22) Filed: Apr. 13, 2009

(51) Int. Cl. A61K 31/60; A61P 9/10

(52) U.S. Cl. 514/161; 514/162

(57) ABSTRACT

As many as 60% of patients who experience an acute coronary syndrome die before they even reach a hospital or other health care facilities, hence the importance of an effective treatment that the patient can self-administer at the first symptom or sign of an acute coronary syndrome. That treatment needs to act rapidly, to be easy to use, to be effective, to be safe, and readily available. The present invention describes a pharmaceutical combination containing a beta blocker agent, one or more anti-platelet agent(s) and a coronary vaso-dilator agent all included in a dose format to be taken orally or by sublingual spray to prevent myocardial infarction.

### FIELD OF THE INVENTION

The present invention is directed to a combination of medications that will stop the evolution of an acute coronary syndrome and prevent the development of a myocardial infarction by acting on 3 of known causative processes; that is

1-coronary vaso constriction,
2-myocardial instability +/−arrhythmias,
3-platelet aggregation with clot formation and coronary thrombosis.

### BACKGROUND TO THE INVENTION

Hundreds of thousands of people die from myocardial infarction annually, the cost of medication and/or surgical interventions on survivors of a myocardial infarction is huge and the loss in human productivity is also very high; that is why a medication that can effectively prevent the evolution of an acute coronary syndrome to a myocardial infarction can be a life saver to many patients. The invention can be self-administered in the pre-hospital setting.

### SUMMARY

A treatment to stop an acute coronary syndrome and prevent its evolution to a myocardial infarction by using a pharmaceutical combination comprising a beta blocker agent, an anti-platelet agent, and a coronary artery dilator in a tablet, to be taken orally or sublingually or by nasal spray, the treatment to be used in the pre-hospital environment.

### DETAILED DESCRIPTION

In the process of coronary artery disease there is a build up of cholesterol in the sub intimal space of the coronary arteries, the cholesterol deposit evolves to the production of atherosclerotic plaques, this process brings about a reduction in the diameter of the coronary artery with a resultant reduced blood flow. Transient vaso constriction of the coronary arteries can also contribute to a reduced blood flow to the myocardium.

Myocardial ischemia is a condition in which there is oxygen deprivation to the heart muscle accompanied by inadequate removal of metabolites because of reduced blood flow or perfusion. Myocardial ischemia can occur as a result of increased myocardial oxygen demand, reduced myocardial oxygen supply, or both. In the presence of coronary obstruction, an increase of myocardial oxygen requirements caused by exercise, tachycardia, or emotion leads to a transitory imbalance. This condition is frequently termed demand ischemia and is responsible for most episodes of chronic stable angina. In other situations, the imbalance is caused by acute reduction of oxygen supply secondary to increased coronary vascular tone (ie, coronary vasospasm) or by marked reduction or cessation of coronary flow as a result of platelet aggregates or thrombi. This condition, termed supply ischemia, is responsible for myocardial infarction (MI) and most episodes of unstable angina (UA). In many circumstances, ischemia results from both an increase in oxygen demand and a reduction in supply.

Pathophysiology: Myocardial ischemia most often develops as a result of reduced blood supply, due to atherosclerotic plaques located in the coronary arteries. The plaques initially allow sufficient blood flow to match myocardial demand. These areas of narrowing may become clinically significant and precipitate angina when myocardial demand increases. Angina that is reproduced by exercise, eating, and/or stress and is subsequently relieved with rest and without recent change in frequency or severity of activity necessary to produce symptoms is called chronic stable angina, patients usually learn to recognize their symptoms. Over time, the plaques may thicken and rupture, exposing a thrombogenic surface upon which platelets aggregate and thrombi form. The lumen of the coronary artery is further reduced and more and more platelets aggregate with the resulting clot formation, the consequence is a marked reduction in coronary blood flow, this syndrome is called “acute coronary syndrome”, resulting in a myocardial syndrome. The symptoms felt by the patient at this time of an impending myocardial infarct (MI) is a retro-sternal chest pain—constrictive in nature, variants exist regarding the location and type of pain, the pain is often accompanied by a sense of fatigue, weakness, and a cold sweat. A myocardial infarct (MI) involves destruction of sections of the heart muscle with all its complications ie. Pump failure, arrhythmias, heart wall rupture, instant death.

That is why it is so important to intervene with an effective treatment before the myocardial infarction takes place. Until recently it was assumed that the death rate from a myocardial infarction was relatively low ie. 15-20% of hospitalized patients. Unfortunately it was realized that close to 60% of patients who experience a syndrome of acute myocardial ischemia or impending myocardial infarction or true myocardial infarction die before they even reach the hospital or other health care facilities, hence the importance of an effective treatment that the patient can self-administer at the first symptom or sign of an acute syndrome of myocardial ischemia. That treatment needs to act rapidly, to be easy to use, to be effective, to be safe, and readily available.

In actual practice:

1. Since there is active vaso constriction of the coronary arteries it is urgent that a beta blocker be used to bring about a Beta-adrenergic vasodilatation; activating Beta-receptors leads to coronary vasodilatation, mediated mostly by a beta1-receptor in conduit arteries and by beta2-receptors in resistance arterioles, thus bringing about a vaso dilation.
2. While plaque fissuring or rupture with platelet aggregation and thrombus formation is a hallmark of unstable angina and Myocardial infarction (Mi), coronary artery constriction is mediated by the paradoxical response of these atheromatous vessels to products of platelet aggregation and thrombosis, namely serotonin, which is released from aggregating platelets. Patients with unstable coronary syndromes and complex plaques have augmented release of serotonin into the coronary circulation. Anti-platelet agent such as Acetylsalicylic acid 80 mg or Clopidogrel 75 mgms, or both medications used jointly can act rapidly and effectively to reduce platelet aggregation and prevent subsequent thrombus formation and obstruction of the coronary arteries by thrombosis and subsequent myocardial infarction.
3. Nitro-glycerin will act as a coronary artery dilating agent.

The pharmaceutical combination of the invention consist of a combination of:
1. Beta blocker such as Metoprolol 50 mgms
2. Anti-platelet agent such as Acetylsalicylic acid 80 mg
3. Coronary vaso dilator such as Nitro-glycerin 0.4 mg
4. Clopidogrel an Anti-platelet agent can also be included as 75 mgs, or replace Acetylsalicylic acid (Aspirin)

The combination of these medications has the following properties:
1. A Beta blocking agent will act as a coronary vaso dilator (cardio-stabilizer) and also reduce risk of Arrhythmia and will improve myocardial pump action.
2. The anti platelet agents will reduce blood coagulability and reduce thrombus formation in either the coronary arteries (or in the cerebral arteries).
3. The coronary vasodilator medication will cause a coronary dilatation thus facilitating coronary artery perfusion.

These 3 or 4 medications can be taken the following ways:
Prepared (tabletized) in such a way so as to be readily solubilized and absorbed and taken sublingually or may be taken orally (P.O.) and swallowed.

These medications could alternatively be taken as a sublingual spray.

Clinically these medications could be used for instance:
1. In patients suffering from coronary heart disease.
   If the patient should have massive anterior chest pain that does not respond to nitro-glycerin sublingual then the patient should take the present pharmaceutical combination.
2. In patients suffering from a transient ischemic attacks.
   In this instance if the patient feels aggravation or progression of his/her neurological symptoms or signs then he/she could take the present medication sublingually, or orally, or as a nasal spray.

Patients should be instructed to take no more than 3 tablets of the pharmaceutical combination herein described or 3 sublingual sprays as a maximum dosage within one hour. The patients should be evaluated by a physician as soon as possible, for proper diagnosis and treatment.

The advantages of the present pharmaceutical combination are obvious; it can be used in the acute coronary syndrome, in the pre myocardial infarction syndrome or as an initial emergency treatment in the case of an acute myocardial infarction.

As noted previously the same pharmaceutical combination can also be used in the case of a Transient Ischemic Attack (TIA), to prevent a cerebro-vascular thrombosis. A process of reduced blood flow to the brain is also involved in that syndrome; reduced blood flow in the cerebral arteries due to reduced arterial lumens partially obstructed by atheromatous plaques, a thrombus may form often accompanied by vaso-constriction which further reduces the blood flow. Often the process is also accompanied by arrhythmias. Ultimately if the process is not stopped a cerebral artery thrombosis or embolism may take place causing brain damage with resultant paralysis and other neurological signs and symptoms. In the case of the Transient ischemic attack the dosage of the medication may be the same or similar as previously described.

The pharmaceutical combination can be self-administered and when used, the incidence of side effects is low and the potential side effects are also minimal when compared with the potential evolution of the process (a myocardial infarction+/−cardiac arrhythmias) in the absence of prompt treatment. Medically speaking the ratio of gain/risk benefit is highly positive.

### Example: 1

The following treatment was used in 22 patients who presented with symptoms and signs of acute myocardial ischemia:

Acetyl-salicylic acid 80 mgs, metoprolol 50 mgs, nitro-glycerin 0.04 mg The pharmaceutical combination was used as an easily dissolved sub-lingual tablet. All of these patients were already known as patients suffering from coronary artery disease. Patients chosen for the treatment were presenting with classical signs of acute coronary syndrome consisting in constrictive chest pain, fatigue, and signs of ST instability on the EKG. 18 patients recovered without evolving to the stage of myocardial infarction. 4 suffered a myocardial infarction (mild) and all patients survived.

### Example: 2

The following pharmaceutical combination was used in 10 patients presenting with symptoms of TIA and 3 patients presenting symptoms and signs of an developing cerebro vascular accident.

Acetyl-salicylic acid 80 mgs, metoprolol 50 mgs, nitro-glycerin 0.04 mg The pharmaceutical combination was used as an easily dissolved sub-lingual tablet. All of these patients were already known as patients suffering from cerebro vascular arteriosclerosis. The patients chosen for the treatment were presenting with very recent symptoms and signs of TIA. 4 patients required 2 doses of the pharmaceutical combination. All patients recovered fully, and were referred to a hospital for further treatment.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Accordingly, the scope of protection is not limited by the description set out above, but is only limited by the claims herein made.

1. A pharmaceutical combination comprising the following: a beta blocking agent such as metoprolol, a coronary vaso-dilating agent such as nitro-glycerin and anti platelet agents such as acetyl salicylic acid (aspirin). Alternately the anti platelet agent can be clopidogrel, or again the anti platelet agent can be a combination of acetyl-salicylic acid (aspirin) and clopidogrel.

2. Wherein the beta blocker is selected from the group consisting of: acebutolol, alprenolol, amosulalol, arotinolol, atenolol, befunolol, betaxolol, bevantolol, bisoprolol, bopindolol, bucumolol, bufetolol, bufuralol, bunitrolol. buprandolol, butofilolol, carazolol, carteolol, carvedilol, celiprolol, cetamolol, cloranolol. dilevalol, epanolol, indenolol, labetalol, levobunolol, mepindolol, metipranolol, metoprolol, moprolol, nadolol, io nadoxolol, nebivalol, nipradilol, oxprenolol, perbutolol. pindolol, practolol, pronethalol, propranolol, sotalol, sufinalol, talindol, tertatolol, tilisolol, timolol, toliprolol, and xibenolol, and pharmaceutically acceptable salts or solvates thereof, or solvates of such salts.

3. The pharmaceutical combination according to claim 1, wherein the beta blocker is metoprolol or a pharmaceutically acceptable salt or solvate thereof. The pharmaceutical combination may use as a beta blocker metoprolol succinate, metoprolol tartrate or metoprolol fumarate.

4. The molar ratio between the beta blocker such as metoprolol and acetyl salicylic acid can lie in the range of 1000:1 and 1:1000.

5. The molar ratio between the beta blocker such as metoprolol and clopidoghrel can lie in the range of 1000:1 and 1:1000.

6. A pharmaceutical combination comprising a beta blocker and acetyl salicylic acid+/−clapidogrel and nitro glycerin in an admixture with a pharmaceutically acceptable adjuvant, diluant or carrier.

7. This pharmaceutical combination is to be used by patients suffering from an acute coronary syndrome, or Impending myocardial infarction for the purpose of stopping the process and preventing a myocardial infarction.

8. This pharmaceutical combination may also be used in patients presenting with signs or symptoms of Transient Ischemic Attack (TIA), an impending cerebro-vascular accident. The syndrome transient ischemic attack, temporary and focal loss of cerebral function, cerebral blood flow reduction, stroke, ischemic stroke, carotid artery atherosclerotic disease, vertebral artery atherosclerotic disease, impending stroke, atherosclerotic disease, vertebral artery stenosis, carotid artery stenosis, cerebral embolism, ventricular thrombus or atrial fibrillation, thromboembolism

9. This pharmaceutical combination can be taken as a tablet orally or sub-lingually, or as a sub-lingual spray; all prepared according to known art.
